(19) United States
(12) Patent Application Publication
(10) Pub. No.: US 2023/0408417 A1
(43) Pub. Date: Dec. 21, 2023

(54) METHOD AND SYSTEM FOR COMPANION ANIMAL HEALTH DIAGNOSIS USING IMAGE OF SAMPLE

(71) Applicant: The LittleCat. Co., Ltd., Gangwon-do (KR)

(72) Inventors: Daeyong KIM, Seoul (KR); Yujeong KIM, Gyeonggi-do (KR); Jihoon PARK, Gyeonggi-do (KR); Hyeongyu LEE, Gyeonggi-do (KR); Sangheon IM, Gyeonggi-do (KR)

(21) Appl. No.: 17/982,642

(22) Filed: Nov. 8, 2022

(30) Foreign Application Priority Data
Jun. 16, 2022 (KR) 10-2022-0116835
Sep. 14, 2022 (KR) 10-2022-0115349

(51) Int. Cl. G01N 21/78; G01N 33/493

(52) U.S. Cl. CPC G01N 21/78; G01N 33/493; G06T 2207/10024

(57) ABSTRACT

A method for a companion animal health diagnosis using an image of a sample, includes: preparing a reference sheet in which a tag formed in a specific pattern is displayed on one surface thereof; acquiring an inspection image including an area of a sample in contact with urine and an area of the tag in a state in which the sample in contact with urine is positioned around the reference sheet using a user terminal; extracting a preset detection area based on the tag from the inspection image and detecting an image of the sample including sample in contact with urine within the detection area; and diagnosing a health status of the companion animal corresponding to a urine status by analyzing a color of the image of the sample.

### BACKGROUND

The present invention relates to a method for a companion animal health diagnosis using an image of a sample, and more particularly, to a method and a system for diagnosing a health status of a companion animal by analyzing a discoloration status of a sample that is discolored according to urine components.

In diagnosing and treating a companion animal's disease, when the symptoms of the disease are insignificant, people do not easily notice it. In addition, when people go to a veterinary hospital and diagnose the presence of a disease, in some cases, a specific disease has already advanced a lot and the recovery thereof is often impossible.

In particular, it may be difficult for people with reduced mobility or the elderly to regularly visit a veterinary hospital to prevent and diagnose diseases of companion animals in advance.

Accordingly, there is a need for a method for periodically checking the health status of the companion animals even at home.

On the other hand, a method of analyzing the health status of the animal using a urine strip whose color is changed by chemical change due to urine is also used.

In the method using the urine strip, a certain amount of urine is immersed in a reagent pad by using a test strip and then, the reagent pad and the colorimetric part are compared with the naked eye.

Since this visual inspection method is not easy for the general public to inspect, a specialist or a separate urine inspection strip analyzer is required. In addition, because the price of the strip analysis device for the urine test is expensive, there is a problem that the general public cannot easily use it.

In order to solve this problem, a technique for analyzing an inspection result by photographing a urine inspection strip with a mobile phone has been proposed in Korean Patent Registration No. 10-2139210 and Korean Patent Registration No. 10-1898594.

In the conventional method, a urine inspection strip having a reagent pad and a colorimetric part is photographed, and the colorimetric part and the color of the discolored reagent pad are compared and analyzed in the photographed image to diagnose the health status.

That is, in the conventional method, the urine excreted by companion animals must be directly collected and tested after the reagent pad is stained with the urine, so that there is a cumbersome procedure for collecting urine. In particular, in the case of a cat excreting in sand, it is difficult to collect the urine.

In addition, since the conventional inspection strip is formed integrally with the reagent pad part and the colorimetric part, whenever urine is analyzed, not only the contaminated reagent pad part but also the colorimetric part must be thrown away at once, so it is difficult to reuse.

Accordingly, Korean Patent Application Laid-Open No. 10-2021-0057842 discloses a technology for constituting an application program to automatically read a photograph taken by a urine inspection unit and provide the inspection result to the user.

However, in the Patent literature, the color of the sample may vary depending on the environment in which the user takes an image or the ambient brightness, and it takes a considerable amount of time to detect the urine area in the application, or an inspection error occurs due to the wrong detection of the urine area.

### PATENT LITERATURE

Patent Literature 1: Korean Patent Registration No. 10-2139210 (Jul. 23, 2020; Mobile urine diagnosis method using multi-color board and strip)
Patent Literature 2: Korean Patent Registration No. 10-1898594 (Sep. 7, 2018; Urine analysis strip and urine analysis method using the same)
Patent Literature 3: Korean Patent Application Laid-Open No. 10-2021-0057842 (May 24, 2021; Pad for companion animals and humans using urine inspection unit and smart automatic reading and confirmation system using the same)

### SUMMARY OF THE INVENTION

The present disclosure has been made in an effort to solve the problems described above, and an objective of the present invention is to provide a method of diagnosing a health status of a companion animal in a simple and accurate way without the cumbersome procedure of putting urine on a reagent pad by analyzing a color of a sample image in an inspection image taken after positioning a reference sheet with a tag formed in a predetermined pattern and a reference color area on one side around the sample in contact with urine.

Another object of the present invention is to set a certain area as a detection area by using a tag as a reference position to improve a detection speed and a detection accuracy of a sample and to allow users to easily recognize a location where a sample is placed.

Further another object of the present invention is to form a tag in a binarized figure pattern, to provide a reference color area inside the figure pattern to easily perfume a white-balance of a sample image, and to use the white-balanced sample image to more accurately diagnose the health status of the companion animals according to the color comparison color thereof.

According to one aspect of the present invention so as to accomplish these objects, there is provided to a method for a companion animal health diagnosis using an image of a sample, including: preparing a reference sheet in which a tag formed in a specific pattern is displayed on one surface thereof; acquiring an inspection image including an area of a sample in contact with urine and an area of the tag in a state in which the sample in contact with urine is positioned around the reference sheet using a user terminal; extracting a preset detection area based on the tag from the inspection image and detecting an image of the sample including sample in contact with urine within the detection area; and diagnosing a health status of the companion animal corresponding to a urine status by analyzing a color of the image of the sample.

At this time, a reference color area is displayed on one surface of the reference sheet and in the diagnosing step of the urine status, when the image of the sample is detected within the detection area, a reference color in the inspection image is compared with a preset reference color value to correct the color of the image of the sample and the health status of the companion animal corresponding to the sample color is diagnosed by analyzing the corrected color of the image of the sample.

In addition, the reference color area is located inside the tag.

In addition, in the image acquisition step, the detection area is displayed on a photographing screen of the user terminal.

In addition, in the image acquisition step, the detection area is displayed on a photographing screen of the user terminal.

In addition, in the image acquisition step, the image of the sample is displayed in the form of a virtual image at a specific location on the detection area.

In addition, the reference color area is formed inside a polygonal outline having a recess and a color of the central area of the polygonal shape is used as a reference color.

In addition, the sample is a bead sample in the form of granules that changes color due to a chemical change when it comes into contact with urine.

In addition, the sample is a defecation pad in which a reagent layer that changes colors depending on urine components is formed on an absorbent sheet for absorbing the urine of the companion animals.

According to another aspect of the present invention so as to accomplish these objects, there is provided to a system for a companion animal health diagnosis using an image of a sample, including: a defecation pad including a reagent layer that changes colors depending on urine components formed on the absorbent sheet for absorbing the urine of companion animals and a tag of a certain pattern having a reference color area printed around the absorbent sheet; a user terminal that takes an inspection image having a urine area and a tag area on the defecation pad, transmits it to a server, and outputs health diagnosis information of the companion animals provided from the server in response thereto; and the server that extracts an image of a sample corresponding to the urine area from the inspection image received from the user terminal, corrects a color of the image of the sample by comparing a color of the reference color area of the tag in the inspection image with a pre-registered reference color value, detects a color of the reagent layer in the urine area from the corrected image of the sample, generates health diagnosis information of the companion animal corresponding to the detected color of the reagent layer, and transmits it to the user terminal.

In addition, the defecation pad is configured to be printed on a bottom surface of the absorbent sheet and the user terminal acquires the inspection image by taking the image in a folded state in order that the tag printed on a bottom surface of the defecation pad is located around the urine area of a top surface of the defecation pad. In addition, the defecation pad is configured by printing first to fourth tags on each corner side of the absorbent sheet and in a state that all of the first to fourth tags of the defecation pad are recognized, the user terminal acquires the inspection image by taking the image including all of the first to fourth tags.

In addition, the server compares an average value of the colors of each reference color area of the first to fourth tags with a pre-registered reference color value to perform a color correction on the corresponding image of the sample.

In addition, the server detects the urine area on the defecation pad and compares a color of the reference color area of the tag closest to a center point of the urine area among the first to fourth tags with the reference color to perform a color correction on the corresponding image of the sample.

In addition, the user terminal detects a tag position through a tag pattern recognition, displays a detection area on a photographing screen thereof by setting the tag position as a reference coordinate and setting a certain area around the tag as the detection area, and takes the inspection image including the tag area and the detection area in a state that the urine area is included in the detection area.

In addition, the reference color area is formed inside a polygonal outline having a recess and a color of the central area of the polygonal shape is used as a reference color.

### BRIEF DESCRIPTION OF THE DRAWINGS

The above and other objects, features and advantages of the present invention will be more apparent from the following detailed description taken in conjunction with the accompanying drawings, in which:

FIG. 1 is a configuration diagram of system for a companion animal health diagnosis using an image of a sample according to a first embodiment of the present invention;

FIG. 2 is an exemplary screen of an application installed in a user terminal of FIG. 1;

FIG. 3 is an exemplary screen of a method of displaying a detection area in a user terminal of FIG. 1;

FIG. 4 is a flowchart illustrating a method for companion animal health diagnosis using an image of a sample according to a first embodiment of the present invention;

FIG. 5 to FIG. 7 are diagrams illustrating a sample photographing method according to a first embodiment of the present invention;

FIG. 8 is another configuration diagram of a system for health diagnosis of a companion animal using a sample according to the present invention;

FIG. 9 is a diagram illustrating a sample image photographing method in the system shown in FIG. 8;

FIG. 10 and FIG. 11 are diagrams for explaining a configuration of a system for a companion animal health diagnosis using an image of a sample according to a second embodiment of the present invention;

FIG. 12 is a diagram for explaining an operation of a system for companion animal health diagnosis using an image of a sample shown in FIG. 10;

FIG. 13 is a view for explaining a configuration of a system for companion animal health diagnosis using an image of a sample according to a second embodiment of the present invention; and

FIG. 14 is a screen illustrating an example of a result of a companion animal health diagnosis according to an embodiment of the present invention.

### REFERENCE SIGNS LIST

10: sample 20: reference sheet
21, 200: tag 30: user terminal
40: server 50: toilet sand
100: defecation pad 110: reagent layer
130: detection area 140: inspection area

### DETAILED DESCRIPTION OF THE PREFERRED EMBODIMENT

Hereafter, the preferred embodiment according to the present disclosure are described in detail with reference to the accompanying drawings.

Since the description of the present invention is a mere embodiment for structural and functional description, it must not be interpreted that the scope of the present invention is limited by the embodiments described in the text. That is, since the embodiments can be variously changed and have various forms, it should be understood that the scope of the invention includes the equivalents for realizing the technical concept. Also, since the specific embodiments do not include all objects and effects presented by the present invention, the scope of the present invention is not limited by them.

Unless differently defined, all the terms used here including technical or scientific terms have the same meaning with what is generally understood by one who has common knowledge in the technical field that this invention belongs to. The terms such as those defined in the dictionary commonly used will be interpreted to have the meanings matching with the meanings in the context of the related technologies. Unless clearly defined in this application, they are not interpreted as ideal or excessively formal meanings.

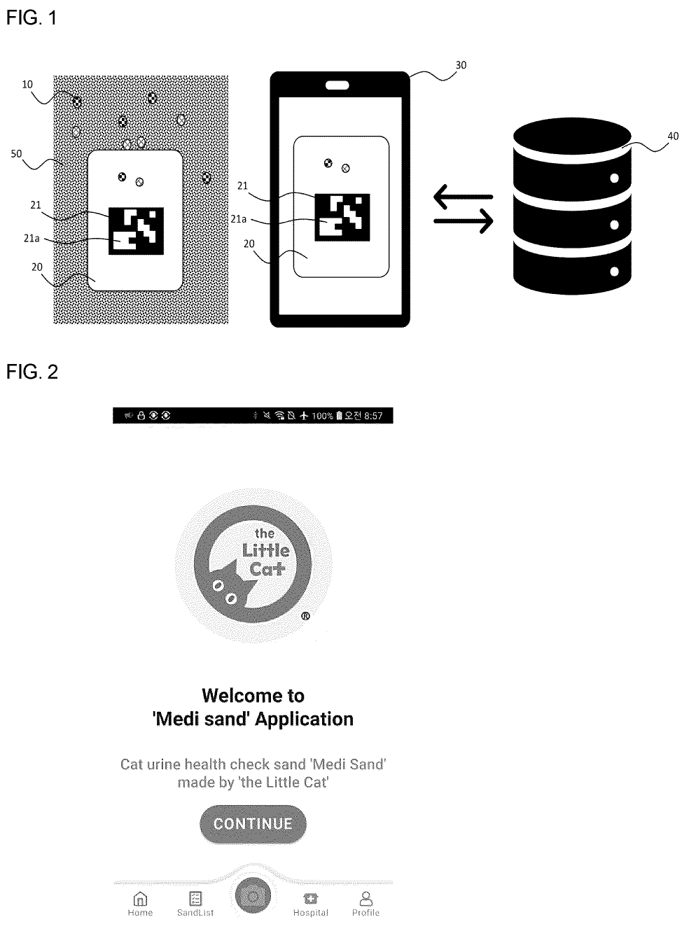

FIG. 1 is a system configuration diagram for a companion animal health diagnosis using an image of a sample according to an embodiment of the present invention.

As shown in FIG. 1, the system for a companion animal health diagnosis using an image of a sample (sample image) includes a sample (10), a reference sheet (20), a user terminal (30), and a server (40).

The sample (10) may be implemented as a bead sample in the form of granules that changes color due to a chemical change when it comes into contact with urine.

At this time, the sample (10) may be sprayed on the urine pad of the companion animal or mixed between the toilet sands (50) and used in such a way that urine comes into contact with the sample.

The reference sheet (20) has a tag (21) formed in a specific pattern on one side thereof and a reference color area.

In this case, the reference color area may be an area other than the tag (21) of the reference sheet (20) or may correspond to a specific pattern formed inside the tag (21).

For example, the reference color area (21a) may be formed in a polygonal shape having a groove and a color of a polygonal central area may be used as a reference color.

In this case, the tag (21) may be implemented as images of various two-dimensional shapes including QR codes.

Also, the tag (21) may be used for the purpose of providing reference coordinates for detecting the image of the sample (10) from the image photographed by the user terminal (30).

The tag (21) is a white polygonal pattern marked on a black background. In this case, the application for examining the sample (10) finds the pattern shape of the tag (21) in the captured(photographed) image and sets a specific position of the pattern shape as a reference point. Also, an area located at a set distance from the corresponding reference point or an area within a set distance from the corresponding reference point may be set as the detection area, so that the sample (10) located within the corresponding detection area may be detected.

The inspection application is to induce the user to position the sample in the detection area by displaying the detection area on the photographing screen of the user terminal (30) in the form of a highlight.

In addition, it is to shorten the image analysis time by image processing only the area set based on the reference coordinates set through the tag (21).

As shown in FIG. 2, an application for health diagnosis of the companion animals is installed in the user terminal (30) and a reference sheet (20) and a camera that takes images in the vicinity of the reference sheet (20) are included.

At this time, the user terminal (30) may include a notebook, a mobile terminal, a smart phone, a tablet PC etc., and all types of cameras equipped with a camera unit capable of taking an image may be included therein.

In addition, the user terminal (30) transmits the image acquired through the camera to the server (40) or provides the user with the diagnosis result transmitted from the server (40).

Accordingly, when the application installed in the user terminal (30) is executed to drive the camera, the user terminal (30) recognizes the pattern of the tag (21) and finds the reference coordinates based on the recognized pattern.

Then, the user terminal (30) displays the detection area (70) of the position set based on the reference coordinates on the photographing screen (60).

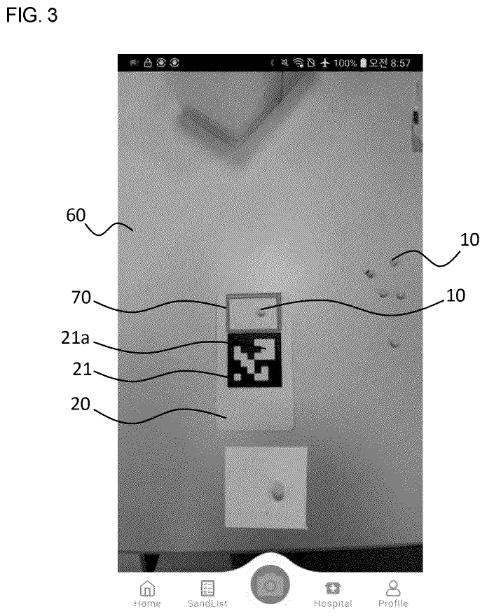

For example, the user terminal (30) displays the detection area (70) in the form of a box at the set position as shown in FIG. 3.

Alternatively, the user terminal (30) may display the image of the sample (10) in the form of a virtual image at a specific location on the detection area based on the reference coordinates, so that the user can easily recognize the location where the sample (10) should be placed.

The server (40) detects the samples in the reference color area and the detection area from the image data received from the user terminal (30) and analyzes the color of the samples to diagnose the health status of the companion animal.

To this end, the server (40) stores data including health status information of the companion animals corresponding to the color values of the samples in contact with urine.

In this case, when the color value of the reference color area is changed due to an external factor, for example, illumination light, it is different from the reference color value, and thus inspection accuracy may be deteriorated.

Accordingly, the server (40) performs white balancing on the image of the sample, so that the color value of the reference color area and the set reference color value become the same.

Then, the server (40) analyzes the color of the sample in the white-balanced sample image to diagnose the health status of the companion animal, and then transmits the result to the user terminal.

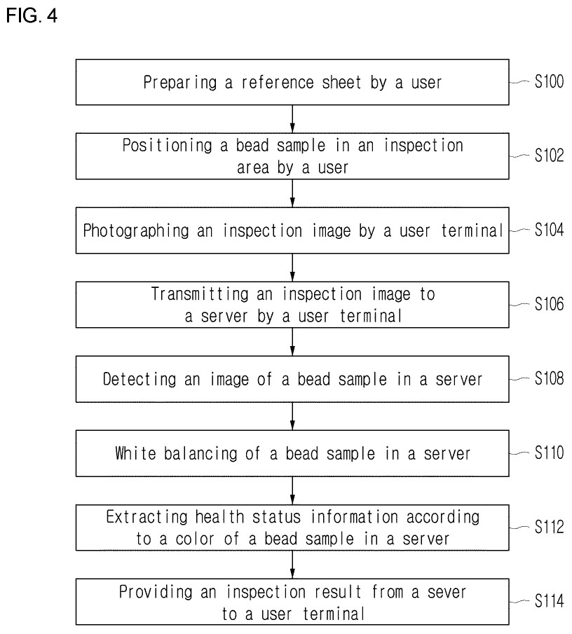

FIG. 4 is a flowchart illustrating a method for companion animal health diagnosis using an image of a sample according to an embodiment of the present invention.

First, the user sprays the sample (10) on a defecation plate or defecation pad so that the urine of the companion animal comes into contact with the sample (10) when the companion animal urinates.

Preferably, the sample (10) is mixed with the toilet sand (50) used as the cat's toilet.

Next, a pre-fabricated reference sheet (20) is prepared (S100) and the side, on which the tag (21) of the reference sheet (20) is foiled, is placed upward, and then the sample (10) in contact with the urine is positioned in the detection area, that is, the upper surface of the reference sheet or in the vicinity of the upper surface of the reference sheet (S102).

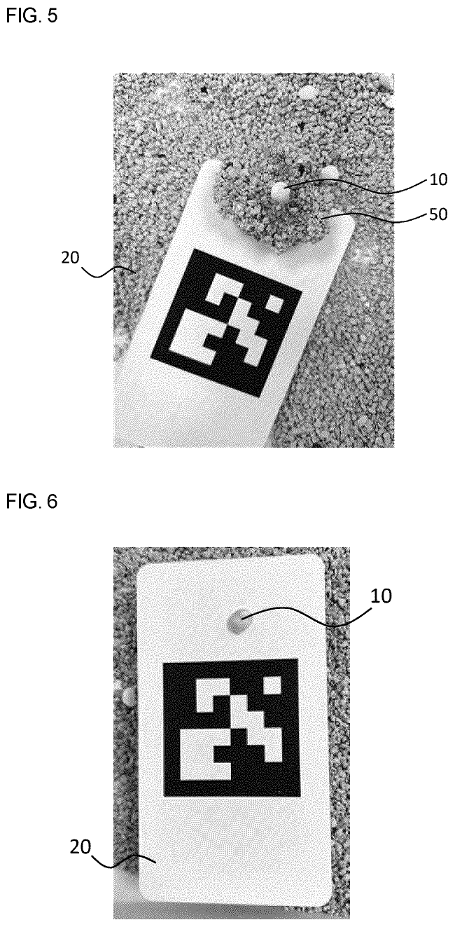

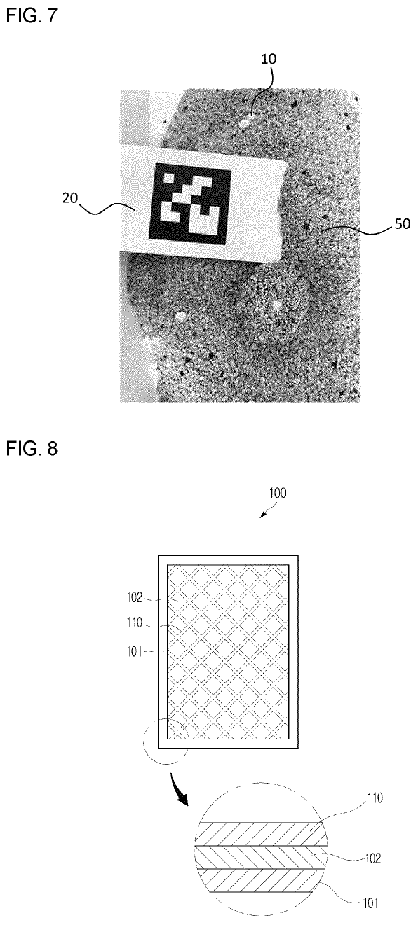

For example, as shown in FIG. 5, the sand (50) and the sample (10) are placed together on the upper surface of the reference sheet (20), or the sample (10) is placed on the upper surface of the reference sheet (20) as shown in FIG. 6, or the sample (10) is placed in the vicinity of the upper surface of the reference sheet (20) as shown in FIG. 7.

Next, when the user terminal (30) runs the application and then drives the camera to photograph the reference sheet (20), the user terminal (30) recognizes the pattern for the reference color area of the tag (21), that is, the white area, on the screen thereof.

Then, the user terminal (30) finds the reference coordinates set based on the recognized pattern, displays the detection area at the position set based on the reference coordinates or displays a virtual bead image in the detection area to induce the sample to be positioned in the detection area.

Next, when the user takes an image while positioning the sample (10) on or near the upper surface of the reference sheet (S104), the user terminal (30) transmits the image to the server (40) (S106). The server (40) detects the image of the sample including the urine-contacted bead-shaped sample (10) within a preset detection area among the inspection images acquired from the user terminal (30) (S108).

In this case, the present invention has an advantage in that the image processing time can be shortened by image processing only the area set based on the reference coordinates detected by the tag (21) to detect the image of the sample.

Then, the server (40) compares the color of the reference color area and a preset reference color in the image, and when there is a difference between the colors, white balancing of the sample image is performed based on the difference value of the colors (S110).

Next, the server (40) analyzes the color information of the sample from the white-balanced sample image, extracts the health status information of the companion animal corresponding to the sample color (S112), and then provides it to the user terminal (30) (S114).

Meanwhile, in the above embodiment, the configuration for acquiring the image of the sample (sample image) in contact with urine using the bead-shaped sample has been described. However, in the present invention, it is also possible to obtain the sample image by using a defecation pad for companion animal interposed with the reagent layer as a sample and to diagnose the health condition of the companion animal by using the sample image.

That is, as shown in FIG. 8, in the present invention, the sample is a defecation pad (100) for companion animals including a waterproof material back sheet (101) and an absorbent sheet (102) for absorbing the urine of the companion animal, which is stacked on the upper surface of the back sheet (101). In addition, a reagent layer (110) that changes colors depending on the components of the urine is foiled on the absorbent sheet (102).

At this time, the defecation pad (100) has a rectangular shape of a predetermined area, and the absorbent sheet (102) having a size smaller than that of the back sheet (101) is disposed on the upper surface of the back sheet (101).

In addition, the reagent layer (110) may be formed by printing an ink of containing an indicator component having a characteristic that is discolored by a urine component on the absorbent sheet (102) in a predetermined pattern or may be formed by laminating a sheet layer of containing the indicator component on the absorbent sheet (102).

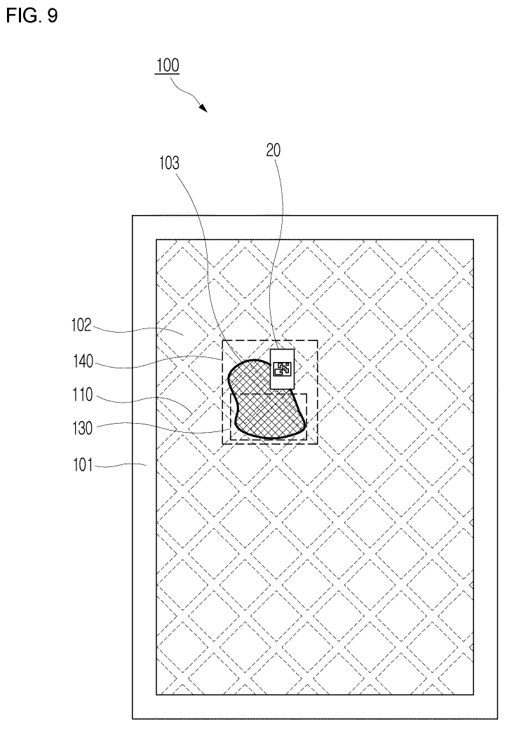

FIG. 9 is a diagram for explaining a method of obtaining a sample image by using the defecation pad (100) having reagent layer (110) shown in FIG. 8 as a sample.

First, when the user places the defecation pad (100) having the reagent layer (110) in the toilet of the companion animal and the companion animal urinates on the defecation pad (100), the surface, on which the tag (21) of the reference sheet (20) is formed, is positioned upward in the vicinity of the urine area (103) of the defecation pad (100).

That is, as shown in FIG. 9, the reference sheet (20) is positioned in such a manner that the urine area (103) is included in the preset detection area (130) around the tag (21).

At this time, when the camera is driven to take an inspection image while the inspection application is executed, the user terminal (30) recognizes a pattern for the reference color area (21a) of the tag (21), that is, the white area, searches for the reference coordinates set based on the recognized pattern, and displays the detection area on the screen of the user terminal (30) at the position set based on the searched reference coordinates. Accordingly, the user may be induced so that the area of the urine-stained reagent layer (110) is located in the detection area (130).

In the above state, when the user takes the image through the user terminal (30), the user terminal (30) acquires the inspection image including the detection area (130) including the reagent layer (110) stained with urine and the inspection area (140) including the area of the tag (21) and transmits it to the server (40).

Then, the server (40) extracts a preset detection area (130) based on the reference coordinates detected by the tag (21) from the inspection images obtained from the user terminal (30) and detects the image of the sample including a reagent layer stained with urine in the detection area (130).

In addition, the server (40) compares the color of the reference color area (21a) of the tag (21) with a preset reference color in the inspection image received from the user terminal (30), and when there is a difference between the colors, the white balancing of the sample image is performed based on the color difference value. In addition, the server may obtain the color information of the reagent layer from a color-corrected sample image through the white balancing, extract health status information of the companion animal corresponding to the color of the reagent layer, and provide it to the user terminal (30).

Meanwhile, in the present invention, when obtaining the sample image using the defecation pad as the sample, it may be carried out to obtain the sample image including the tag by printing the tag on the defecation pad without using the reference sheet.

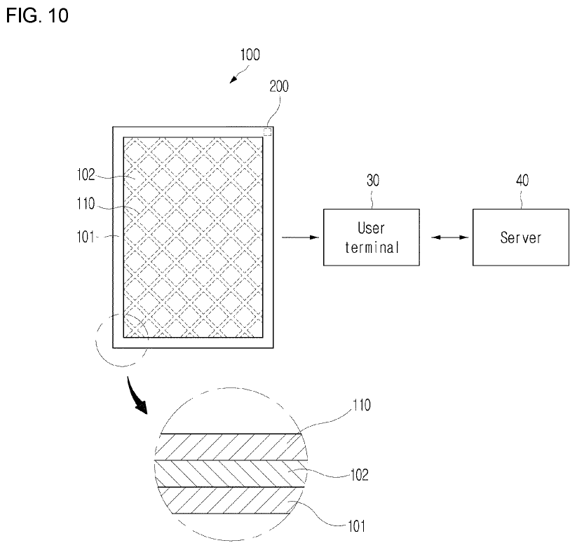

FIG. 10 is a configuration diagram of a system for health diagnosis of companion animals using an image of a sample according to a second embodiment of the present invention.

As shown, a system for health diagnosis of companion animals using an image of a sample includes a defecation pad (100), a user terminal (30), and a server (40).

The defecation pad (100) is a defecation pad for pets having a back sheet (101) of a waterproof material and an absorbent sheet (102) for absorbing the urine of the pet, which is stacked on the upper surface of the back sheet (101). In the present invention, it is configured by forming a reagent layer (110) that changes color depending on the components of urine on the absorbent sheet (102) and printing a tag (200) having a specific pattern on one side of the bottom surface of the back sheet (101).

In addition, the reagent layer (110) may be formed by printing an ink of containing an indicator component having a characteristic that is discolored by a urine component on the absorbent sheet (102) in a predetermined pattern or may be formed by laminating a sheet layer of containing the indicator component on the absorbent sheet (102).

In addition, the tag (200) is printed on the bottom side of the absorbent sheet (102), more specifically, on the bottom side of the back sheet (101) located at the corner thereof.

The tag (200) may be used for the purpose of providing reference coordinates for detecting the image of the reagent layer (110) from the image captured by the user terminal (30).

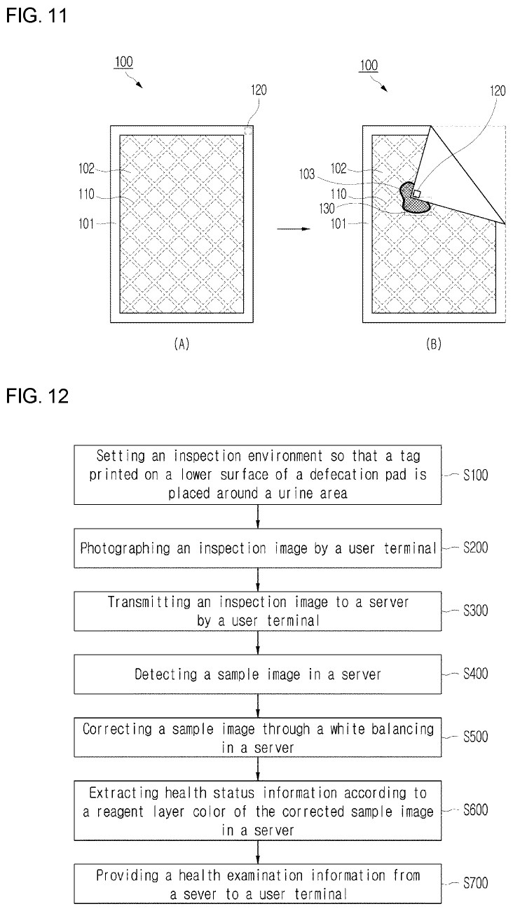

That is, as shown in FIG. 11, the tag (200) is formed on the bottom surface of the defecation pad (100). Accordingly, after folding the portion where the tag (200) of the defecation pad is formed, in a state in which it is positioned close to the urine area (103) on the upper surface of the absorbent sheet (102), the inspection application may set a certain area including the urine area (103) around the tag (200) as the detection area (130).

The user terminal (30) transmits the image acquired through the camera to the server (40) or provides the companion animal health diagnosis result information transmitted from the server (40) to the user.

That is, when the camera is driven by executing the inspection application installed in the user terminal (30), the user terminal (30) recognizes the pattern of the tag (200) and finds reference coordinates based on the recognized pattern.

Then, the user terminal (30) displays the detection area (130) at a position set based on the reference coordinates on the photographing screen to induce the reagent layer (110) with the urine to be included in the detection area (130). For example, the user terminal (30) may display the detection area (130) in the form of a box.

In addition, the user terminal (30) sets the reagent layer (110) on which urine is stained, that is, the detection area (130) including the urine area and the inspection area (140) including the area of the tag (110). The inspection image is obtained by photographing the inspection area (140).

Meanwhile, in FIG. 10, the server (40) detects a sample area corresponding to the reagent layer (110) in contact with the urine within the detection area (130) of the inspection image received from the user terminal (30) and analyzes the color of the reagent layer (110) in the image of the sample, that is, the sample image of the sample area to diagnose the health status of the companion animal.

To this end, the server (40) stores data including health status information of the companion animals corresponding to the color values of the samples in contact with urine.

In addition, the server (40) detects the reference color area of the tag (200) and performs white balancing on the sample image so that the reference color value and the set reference color value are the same.

In this case, when the color value of the reference color area is changed due to an external factor, for example, illumination light, it is different from the reference color value, and thus inspection accuracy may be deteriorated.

Accordingly, the server (40) analyzes the color of the reagent layer in the white-balanced sample image to diagnose the health status of the companion animal, and then transmits the result to the user terminal (30).

FIG. 12 is a flowchart for explaining an operation of a system for the companion animal health diagnosis system using the sample taken image shown in FIG. 10.

First, in a state that the user places the defecation pad (100) having the tag (200), which is printed on the lower surface thereof, on a place of defecation of the companion animal, when the companion animal urinates on the defecation pad (100), the printed portion of the tag (200) is folded to be positioned on the upper surface of the defecation pad (100), and the tag (200) is placed around the area where the urine is stained (S100). That is, the inspection environment is set so that the defecation pad (100) has a shape such as that of FIG. 11 (B).

Then, when the user terminal (30) executes the inspection application and drives the camera to take the inspection image, the user terminal (30) recognizes a pattern for the reference color area of the tag (200), that is, the white area on the screen thereof.

Then, the user terminal (30) finds the reference coordinates set based on the recognized pattern, displays the detection area at the position set based on the reference coordinates. Accordingly, the user may be induced so that the area of the urine-stained reagent layer (110) is located in the detection area (130).

In the above state, when the user takes an inspection image through the user terminal (30) (S200), the user terminal (30) transmits the image to the server (40) (S300).

In this case, the inspection image is a photographed image of the inspection area (140) including the detection area (130) and the area of the tag (200).

The server (40) detects a sample image including a reagent layer stained with urine in a preset detection area (130) from the captured images acquired from the user terminal (30) (S400).

In this case, the present invention has an advantage in that the image processing time can be shortened by image processing only the detection area set based on the reference coordinates detected by the tag (200) to detect the sample image.

Then, the server (40) compares the color of the reference color area (120a) of the tag (200) with a preset reference color in the inspection image received from the user terminal (30), and when there is a difference between the colors, white balancing of the sample image is performed based on the difference value of the color, so that the color correction processing is performed on the sample image (S500).

Then, the server (40) obtains and analyzes reagent layer color information from the color-corrected sample image, extracts the stored health state information corresponding to the color of the reagent layer color (S600), and provides it to the user terminal (30) (S700).

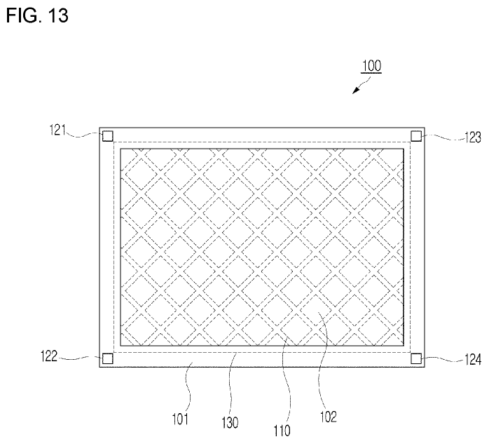

On the other hand, according to the above embodiment, by printing a tag on the bottom surface of the defecation pad and placing the tag around the reagent layer stained with urine in a simple way of folding the defecation pad, the inspection image area is set. However, in the present invention as shown in FIG. 13, the first to fourth tags (201, 202, 203, and 204) are printed and configured on four corners of the upper surface of the defecation pad (100). Therefore, the inspection application, which is installed in the user terminal (30) can be carried out to photograph the entire area of the defecation pad (100) including the reagent layer (110), which is stained with urine, when all the first to fourth tags (201, 202, 203, and 204) are recognized.

At this time, when all of the patterns of the first to fourth tags (201, 202, 203, and 204) are detected, the user terminal (30) sets the detection area (130) based on the first to fourth tags (201, 202, 203, and 204) and takes the inspection image including the detection area (130) and the first to fourth tags (201, 202, 203, and 204).

Then, the server (40) detects the sample image corresponding to the reagent layer (110) on which urine is stained in the detection area (130) and provides health state information corresponding to the color of the reagent layer to the user terminal (30).

At this time, the server (40) compares the color of the reference color area formed in the first to fourth tags (201, 202, 203, and 204) with the pre-registered reference color to perform the white balancing on the sample image based on the difference value between the colors. In this case, by comparing the average value of the colors of each reference color area of the first to fourth tags (201, 202, 203, and 204) with the reference color value, the white balancing may be performed on the sample image.

In addition, the server (40) detects the urine area on the defecation pad (100), selects the tag of the position closest to the center point of the urine area among the first to fourth tags (201, 202, 203, and 204), and then compares the color of the reference color area with the previously registered reference color, so that it is also possible to perform the white balancing on the sample image.

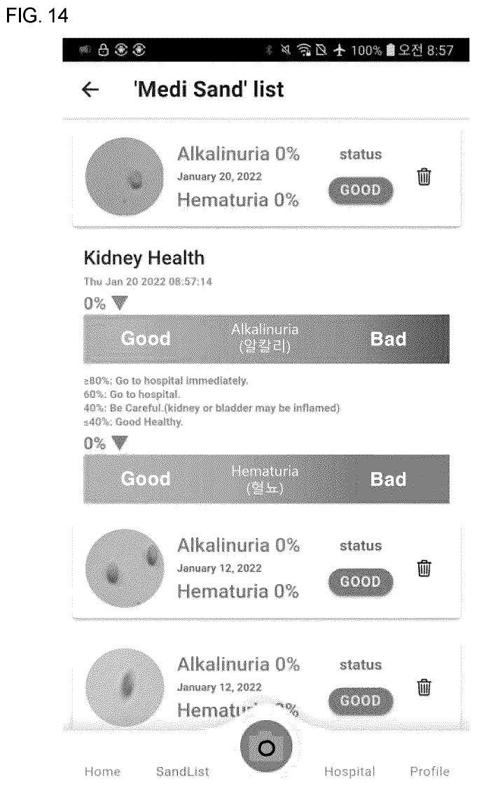

FIG. 14 is a screen illustrating an example of a companion animal health diagnosis result according to an embodiment of the present invention.

As shown, the health status of the kidneys can be provided with information such as alkali level and hematuria level.

In addition, FIG. 14 discloses a form of diagnosing and providing the status of alkaline urine and hematuria in the form of percentage and good or bad.

In addition, the inspection results may be stored in the user's diary app or in the diary menu in the application for each inspection time and date to support the health management of the companion animal.

As described above, according to the present invention, the health status of the companion animal can be easily and accurately checked using the sample image in contact with the urine without a separate urine collection process, so that the health status of the companion animal can be checked and managed at any time.

The present invention has the effect that it is possible to accurately diagnose the health status of the companion animals by automatically analyzing the captured images without purchasing a separate urine inspection strip analysis device and does not require a separate cumbersome procedure for collecting the urine.

In addition, in the case of a cat excreted in sand, since only a bead-type sample that comes into contact with urine is replaced and used, the reference sheet for inspection can be reused, thereby reducing the cost of inspection.

That is, according to the present invention, since even the general public who is not an expert can accurately check the health status of the companion animals in a simple way, there are advantages in that costs can be reduced and the health of the companion animals can be easily managed.

While the present invention has been described with respect to the specific embodiments, it will be apparent to those skilled in the art that various changes and modifications may be made without departing from the spirit and scope of the invention as defined in the following claims.

What is claimed is:

1. A method for a companion animal health diagnosis using an image of a sample, comprising:
preparing a reference sheet in which a tag formed in a specific pattern is displayed on one surface thereof;
acquiring an inspection image including an area of a sample in contact with urine and an area of the tag in a state in which the sample in contact with urine is positioned around the reference sheet using a user terminal;
extracting a preset detection area based on the tag from the inspection image and detecting an image of the sample including sample in contact with urine within the detection area; and
diagnosing a health status of the companion animal corresponding to a urine status by analyzing a color of the image of the sample.

2. The method according to claim 1, wherein a reference color area is displayed on one surface of the reference sheet and in the diagnosing step of the urine status, when the image of the sample is detected within the detection area, a reference color in the inspection image is compared with a preset reference color value to correct the color of the image of the sample and the health status of the companion animal corresponding to the sample color is diagnosed by analyzing the corrected color of the image of the sample.

3. The method according to claim 2, wherein the reference color area is located inside the tag.

4. The method according to claim 1, wherein in the image acquisition step, the detection area is displayed on a photographing screen of the user terminal.

5. The method according to claim 2, wherein in the image acquisition step, the detection area is displayed on a photographing screen of the user terminal.

6. The method according to claim 4, wherein in the image acquisition step, the image of the sample is displayed in the form of a virtual image at a specific location on the detection area.

7. The method according to claim 1, wherein the reference color area is formed inside a polygonal outline having a recess and a color of the central area of the polygonal shape is used as a reference color.

8. The method according to claim 2, wherein the reference color area is formed inside a polygonal outline having a recess and a color of the central area of the polygonal shape is used as a reference color.

9. The method according to claim 1, wherein the sample is a bead sample in the form of granules that changes color due to a chemical change when it comes into contact with urine.

10. The method according to claim 1, wherein the sample is a defecation pad in which a reagent layer that changes colors depending on urine components is formed on an absorbent sheet for absorbing the urine of the companion animals.

11. A system for a companion animal health diagnosis using an image of a sample, comprising:
a defecation pad including a reagent layer that changes colors depending on urine components formed on the absorbent sheet for absorbing the urine of companion animals and a tag of a certain pattern having a reference color area printed around the absorbent sheet;
a user terminal that takes an inspection image having a urine area and a tag area on the defecation pad, transmits it to a server, and outputs health diagnosis information of the companion animals provided from the server in response thereto; and
the server that extracts an image of a sample corresponding to the urine area from the inspection image received from the user terminal, corrects a color of the image of the sample by comparing a color of the reference color area of the tag in the inspection image with a pre-registered reference color value, detects a color of the reagent layer in the urine area from the corrected image of the sample, generates health diagnosis information of the companion animal corresponding to the detected color of the reagent layer, and transmits it to the user terminal.

12. The system according to claim 11, wherein the defecation pad is configured to be printed on a bottom surface of the absorbent sheet and the user terminal acquires the inspection image by taking the image in a folded state in order that the tag printed on a bottom surface of the defecation pad is located around the urine area of a top surface of the defecation pad.

13. The system according to claim 11, wherein the defecation pad is configured by printing first to fourth tags on each corner side of the absorbent sheet and in a state that all of the first to fourth tags of the defecation pad are recognized, the user terminal acquires the inspection image by taking the image including all of the first to fourth tags.

14. The system according to claim 13, wherein the server compares an average value of the colors of each reference color area of the first to fourth tags with a pre-registered reference color value to perform a color correction on the corresponding image of the sample.

15. The system according to claim 13, wherein the server detects the urine area on the defecation pad and compares a color of the reference color area of the tag closest to a center point of the urine area among the first to fourth tags with the reference color to perform a color correction on the corresponding image of the sample.

16. The system according to claim 12, wherein the user terminal detects a tag position through a tag pattern recognition, displays a detection area on a photographing screen thereof by setting the tag position as a reference coordinate and setting a certain area around the tag as the detection area, and takes the inspection image including the tag area and the detection area in a state that the urine area is included in the detection area.

17. The system according to claim 13, wherein the user terminal detects a tag position through a tag pattern recognition, displays a detection area on a photographing screen thereof by setting the tag position as a reference coordinate and setting a certain area around the tag as the detection area, and takes the inspection image including the tag area and the detection area in a state that the urine area is included in the detection area.

18. The system according to claim 11, wherein the reference color area is foiled inside a polygonal outline having a recess and a color of the central area of the polygonal shape is used as a reference color.
